(19) United States
(12) Patent Application Publication
(10) Pub. No.: US 2013/0297028 A1
(43) Pub. Date: Nov. 7, 2013

(54) LOCKING BONE PLATE

(71) Applicant: ARTHRODISC, L.L.C., Park City, UT (US)

(72) Inventor: Richard I. Zipnick, Park City, UT (US)

(21) Appl. No.: 13/667,539

(22) Filed: Nov. 2, 2012

Related U.S. Application Data
(60) Provisional application No. 61/554,589, filed on Nov. 2, 2011.
(60) Provisional application No. 61/554,600, filed on Nov. 2, 2011.
(60) Provisional application No. 61/554,616, filed on Nov. 2, 2011.

(51) Int. Cl. A61F 2/44

(52) U.S. Cl. CPC A61F 2/442
USPC 623/17.16

(57) ABSTRACT

An implant can include: a body having one or more apertures; and one or more tabs associated with the one or more apertures, each tab being movable with respect to the body and one of the apertures. In one aspect, the implant can include: a first position with a first tab in a relaxed state adjacent to a first aperture; and a second position with the first tab a strained state with a portion of the first tab bent away from or toward the first aperture. In one aspect, the implant can include: a first position with a first tab in a relaxed state between at least a first aperture and a second aperture; and a second position with the first tab a strained state with a portion of the first tab bent away from the first aperture and toward the second aperture.

### CROSS-REFERENCE TO RELATED APPLICATIONS

This patent application claim the benefit of U.S. Provisional Application Ser. No. 61/554,589, filed Nov. 2, 2011, U.S. Provisional Application Ser. No. 61/554,600, filed Nov. 2, 2011, U.S. Provisional Application Ser. No. 61/554,616, filed Nov. 2, 2011, which provisional applications are each incorporated herein by specific reference in their entirety.

### BACKGROUND

An intervertebral disc is a soft tissue compartment connecting the vertebra bones in a spinal column. Each healthy disc consists of two parts, an outer annulus fibrosis (hereinafter “the annulus”) and an inner nucleus pulposes (hereinafter “the nucleus”). The annulus completely circumscribes and encloses the nucleus. The annulus is connected to an adjacent associated pair of vertebrae by collagen fibers.

The intervertebral disc is an example of a soft tissue compartment adjoining first and second bones (vertebras) having an initial height and an initial width. Other joints consisting of a soft tissue compartment adjoining at least first and second bones having an initial height and an initial width include the joints of the hand, wrist, elbow, shoulder, foot, ankle, knee, hip, etc.

Typically, when a disc is damaged, the annulus ruptures and the nucleus herniates. Discectomy surgery removes the extruded nucleus, leaving behind the ruptured annulus.

The ruptured annulus is, by itself, ineffective in controlling motion and supporting the loads applied by the adjacent pair of vertebrae. With time, the disc flattens, widens, and bulges, compressing nerves and producing pain. Uncontrolled loads are transmitted to each vertebra. Each vertebra tends to grow wider in an attempt to distribute and compensate for higher loads. When a vertebra grows, bone spurs form. The bone spurs further compress nerves, producing pain.

A variety of expandable intervertebral devices are disclosed in the art to replace the intervertebral disc. Such devices are implanted intermediate an adjacent pair of vertebra, and function to assist the vertebra. These devices do not assist the intervertebral disc. In fact, in many cases the disc is removed.

Prior art intervertebral devices are either static or dynamic. A static intervertebral device eliminates motion. Static devices are generally square, rectangular, trapezoidal, or box shapes that are immobile. Static devices replace the disc to facilitate bone fusion. The insertion of a static device requires near total removal of the disc. An adjacent pair of vertebrae ordinarily is contoured to the static device and a bone graft. A static device temporarily maintains the vertebrae immobilized until the bone graft heals. Static devices may, on insertion, initially expand, but their final state is immobile. Core elements with the threads on one portion reversed or oppositely wound from threads on another portion have been frequently utilized to expand immobilization (fusion) devices.

Dynamic devices are configured to be capable of moving. Inserting a dynamic device, such as a total disc prosthesis, requires a near total removal of disc tissue. A dynamic device ordinarily is inserted to contour to the vertebral bones without a bone graft. Usually the vertebral bones are contoured to the dynamic device. Round, curved, or circular shaped devices inserted after removing disc tissue or vertebral bone tend to migrate in the intervertebral disc space or subside within the vertebral bone. Dynamic devices are often permanent devices that replace a disc, connect vertebral bones together, and allow movement. Dynamic devices initially may expand. Also, their final state can be mobile.

Other dynamic devices require partial removal of disc tissue. These devices can be inserted within the interior (nucleus) of an intervertebral disc and contour to the vertebral bones. Nucleus devices are generally smaller than devices used as a total disc prosthesis. Nucleus devices often are single part locking mechanisms. Fixation generally is not used and the device typically migrates within the disc space or subsides in vertebral bones. Other dynamic devices do not have a solid bearing surface, but include liquid or gas.

Other devices and methods function to patch or seal a disc without substantially supporting the vertebra. Inserting these devices requires the removal of disc tissue. These devices are usually added to the annulus, which causes widening of the annulus, and the device increases the risk of contacting the nerves of the spinal column when the disc is compressed. Still other devices can form a physical barrier with the annulus in order to function, where such a barrier positioned within the annulus can prevent the annulus from healing. Still other devices change the material property of the disc.

Therefore, there remains a need in the art for improved devices and methods for treating injuries, deformations, or other defects in any of the intervertebral discs of the spine.

### SUMMARY

In one embodiment, an implant can include: a body having one or more apertures; and one or more tabs associated with the one or more apertures, each tab being movable with respect to the body and one of the apertures. In one aspect, the implant can include: a first position with a first tab in a relaxed state adjacent to a first aperture; and a second position with the first tab in a strained state with a portion of the first tab bent away from or toward the first aperture. In one aspect, the implant can include: a first position with a first tab in a relaxed state between at least a first aperture and a second aperture; and a second position with the first tab in a strained state with a portion of the first tab bent away from the first aperture and toward the second aperture, or vice versa. The second position can include the tab over or otherwise reducing the openness of the second aperture.

In one embodiment, the tab can include a base connected to the body, a stem connected to the base, and a head connected to the stem opposite of the base. In one aspect, the head can be wider than the stem. In one aspect, the head can have a first lobe defining a portion of a first aperture and as second lobe defining a portion of a second aperture. Optionally, the first and second lobes can be hemispheres of a circular head, or the lobes may protrude laterally toward and/or into the apertures such that the head is oblong. In one aspect, the tab can include a groove through the head and at least a portion of the stem so as to form adjacent tab flaps. In one aspect, the adjacent tab flaps can include a first tab flap defining a portion of a first aperture and a second tab flap defining a portion of a second aperture.

In one embodiment, the tab can be integrated with the implant, where the tab and implant are formed of the same material and/or from the same member (e.g., flat plate member). In one embodiment, the tab is cut out from the body of the implant, where the cut can be by laser, water jet, or saw cutting or other cutting into the body to form the shape of the tab therefrom.

In one embodiment, the implant can include first and second apertures that include first and second slotted grooves adjacent to a stem of the tab. In one aspect, slotted apertures are at an end of the slotted groves opposite of the first and second apertures. The slotted grooves and/or slotted apertures facilitate movement of the tab, such as pivoting about the base. The slotted apertures can be adjacent to the base so as to create a smaller cross-sectional profile at the base to facilitate pivoting thereabout. Similarly, the slotted grooves can allow the stem to bend, and the arrangement of the slotted apertures can define the shape and dimensions of the stem, which can be varied to vary the shape and dimension of the stem.

In one embodiment, the implant includes a wall defining the first and second apertures and the tab therebetween. The wall can be continuous around the first and second apertures. The continuous wall of the first and second apertures can form a major aperture or larger aperture having the first and second apertures if the tab is removed or lifted out of the plane of the implant. The wall can be sectioned, but continuous. In one aspect, the implant can include a wall defining first and second apertures and the tab therebetween and the groove in the tab that forms the tab flaps. Similarly, the wall can be continuous, but may also have sections.

In one embodiment, the body is a unitary member with the tab formed therein, wherein at least one aperture defines a portion of the tab and a portion of the tab defines a portion of an aperture. In one aspect, the body is a unitary member with the tab formed therein, and wherein at least two apertures define two opposite sides of a head of the tab. In one aspect, the body is a unitary member with the tab formed therein, wherein at least two apertures define two opposite two sides of a head of the tab, and wherein an end groove extends between the two apertures and defines an end of the tab between the two sides. In one aspect, the body is a unitary member with the tab formed therein, wherein at least two apertures define two opposite sides of a head of the tab, wherein an end groove extends between the two apertures and defines an end of the tab between the two sides, and wherein a slotted grove extends from each of the two apertures so as to form a stem of the tab. In one aspect, the body is a unitary member with the tab formed therein, wherein at least two apertures define two opposite sides of a head of the tab, wherein an end groove extends between the two apertures and defines an end of the tab between the two sides, and wherein a slotted groove extends from each of the two apertures so as to form a stem of the tab, the tab has a base connected to a main body portion of the body, the stem is connected to the base, and the head is connected to the stem opposite of the base. In one aspect, the body is a unitary member with the tab formed therein, wherein at least two apertures define two opposite sides of a head of the tab, wherein an end groove extends between the two apertures and defines an end of the tab between the two sides, and wherein a slotted groove extends from each of the two apertures so as to form a stem of the tab, the tab has a base connected to a main body portion of the body, the stem is connected to the base, and the head is connected to the stem opposite of the base, the apertures, end groove, and slotted grooves are defined by a continuous wall of the body.

In one embodiment, a method of manufacture can be used for forming the implant having the apertures and tabs as described herein. Such a method can include: providing the body; forming the one or more apertures in the body; and forming the one or more tabs. In one aspect, the method can include: providing the body; and forming the apertures, end groove, and slotted grooves so as to form the tabs. In one aspect, the thickness from a tab can be thinned compared to the thickness of the bone plant implant. The thickness can be from a top planar side to a bottom planar side.

In one embodiment, an implant system can include the implant having the apertures and tabs as described herein, and one or more fasteners dimensioned to fit in the one or more apertures. In one aspect, the first fastener includes a fastener head that presses against a first tab when inserted into an aperture that defines a surface of the first tab. In one aspect, the implant system can include one or more cams dimensioned to fit in a portion of the one or more apertures. In one aspect, both the fasteners and cams can be included or only one of the fastener or cam can be included in a kit of the system. In one aspect, a first fastener includes a first fastener head that moves a first tab when inserted into a first aperture that defines a surface of the first tab, and a first cam includes a second fastener head that presses against the first tab when inserted into a second aperture that defines a surface of the first tab and the first cam moves the first tab toward the first aperture when the first cam is rotated.

In one embodiment, an implant as described can be mounted to bone. A method of mounting an implant to a bone can include: providing the implant or implant system as described herein; inserting a first fastener into a first aperture so as to move a head of the first tab; and inserting the first fastener into the bone so as to fasten the implant to the bone. In one aspect, the method can include: rotating the head of the first tab away from the first aperture toward a second aperture before or during inserting the first fastener into the first aperture; and rotating the head of the first tab toward the second aperture before or during inserting a second fastener into the second aperture. In one aspect, the method can include pressing the head of the first tab against the first fastener so as to friction couple the first tab with the first fastener. In one aspect, the method can include pressing the head of the first tab against the first fastener and second fastener so as to friction couple the first tab with the first fastener and second fastener. In one aspect, the method can include covering at least a portion of the first fastener with the first tab. In one aspect, the method can include covering at least a portion of the first fastener and second fastener with the first tab. In one aspect, the head of the first tab can be lifted from the plane of the implant, and placed over the first fastener in the first aperture and/or second fastener in the second aperture or cam in the second aperture.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 includes a perspective view of an embodiment of a locking bone plate implant with flexible tabs and apertures.

FIG. 1A includes a front view of an embodiment of a flexible tab having an aperture of a locking bone plate implant.

FIG. 2 includes a side view of an embodiment of a locking bone plate implant with a screw for attachment to a vertebra and with a cam locking mechanism.

FIGS. 2A-2D include top views (FIGS. 2A-2C) and side views (FIG. 2D) of cross-section profiles of cams that can actuate the cam locking mechanism of FIG. 2.

FIG. 3 includes a front view of an alternative embodiment of a locking bone plate implant with flexible tabs and apertures.

FIG. 4 includes a front view of an alternative embodiment of a locking bone plate implant with flexible tabs and apertures.

FIG. 5 includes a front view of an alternative embodiment of a locking bone plate implant with flexible tabs and apertures.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to bone plates having flexible tabs and apertures associated and/or at least partially defined by the flexible tabs. The tabs can be flexible so as to move a head of the tab relative to one or more apertures in the bone plate.

The invention also relates to methods of manufacture, implantation, extraction, or other uses of the bone plates. Common uses of the bone plate can be therapeutic by implantation onto one or more bones for stabilization thereof, such as to stabilize a fracture of a bone. Examples can include a spinal plate, blade plate, buttress plate, compression plate, neutralization plate, pressure plate, reconstruction plate, and like orthopedic plates. The spinal plate embodiment can be used for stabilizing one or more vertebras, and can be mounted to a single vertebra or two or more adjacent vertebras in order to stabilize and facilitate healing of fractures, disc herniations, fusions, inhibition of fusions, and other common spinal plate uses. In some instances, spinal plates can be referred to as lumbar plates, anterior lumbar, or lateral plates. The blade plate embodiment can be shaped at an oblique or right angle for subtrochanteric or supracondylar fractures of the femur, used to bridge a femoral osteotomy, where for example one arm has a chisel-shaped end driven into the bone, bridging the fracture and the other arm is used as a side plate and anchored to the bone with multiple screws. The buttress plate embodiment can be used to support internal fixation of a bone fracture, and may be a thin, flat plate to provide support in surgical repair of fracture, which may be useful for supporting a bone that is unstable in compression or axial loading, such as at the distal radius and tibial plateau to hold impacted or depressed bone fragments in position. The compression plate embodiment can be used to stabilize fractures with lag screws to provide dynamic compression on the tension side of bone, and may have oval screw holes with a beveled floor and an inclined surface, to approximate the ends of the bone as the screws tighten. An embodiment of a neutralization plate can used for the internal fixation of a long bone fracture with lag screws to neutralize the forces producing displacement so as to protect fracture surfaces from normal bending, rotation and axial loading force. The pressure plate embodiment can be configured to apply pressure to bone or fracture thereof to promote healing. The reconstruction plate can have various shapes for reconstructions of fractured or multi-fragmented bone pieces, where the plate may have multiple surfaces at different angles to provide proper bone alignment. Any of these plate embodiments can be configured for application to the spine, such as at adjacent vertebras. Also, the plates can be used to treat spinal deformities, trauma, degenerative lumbar, or the like for treatment of cervical spine applications, anterior column support, posterior applications, or any other. The plates can be used for attachment to one vertebra or to two or more adjacent vertebras.

The bone plates can have one or more of apertures and associated flexible tabs, which apertures can be dimensioned for receiving fasteners, such as bone screws. The apertures can be located at any position on the bone plates, such as in the middle or at ends. Each aperture has a tab that can be flexed so as to be bent away from and/or toward the aperture. For example, a head of the tab can be moved by bending a stem of the tab, which stem allows for the head to freely pivot with respect to a pivot point at the base of the stem. A single tab can be associated with two apertures such that bending the tab away from one aperture bends the tab toward the other aperture. A single tab can be located between two apertures so that a head of the tab defines portions of the adjacent apertures, such as defining adjacent or facing portions of the adjacent apertures. Also, a single tab can be positioned between adjacent apertures such that removal of the tab can cause the two adjacent apertures to combine into a single larger aperture. The tab can be coupled or integrated with the body so that a single larger aperture is divided into two smaller apertures with the tab therebetween. The adjacent apertures separated by the tab can include a groove or slit therebetween, which groove or slit contours with the tab head. Often, the bone plate can include one or more apertures on each end of bone plate so that both ends can be fastened to bone. The bone plate may also include a center aperture for fastening to bone or to an intervertebral implant.

The bone plates generally are flat or planar structures with various lengths and widths with a thickness that is usually thinner than the length or width. The thinner thickness allows the bone plate to fit on a bone with a low profile so that the body of the bone plate does not extend too far from the surface of the bone. The tab of the bone plate can have the same thickness as the body or it can be thinner. The thickness of the tab can be from about 10% to 100%, 20% to 90%, 30% to 80%, 40% to 70%, or 50% to 60% of the thickness of the plate or any value therebetween or can be greater than or equal to any of these values.

The bone plate can include apertures that are configured to receive screws therethrough for affixation to a bone surface. The apertures can be of various sizes, cross-sectional profiles, and configurations (e.g., threaded or smooth) to receive various types of fasteners, such as bone screws. The apertures include or are partially defined by flexible tabs, which can flex during insertion of a screw with resiliency to press back onto the screw, and thereby the flexible tabs can press against the screw in order to inhibit the screw from being unscrewed from the bone and/or from the bone plate. The apertures can have a cross-sectional profile that can be circular, however, the cross-sectional profile can include a portion that is indented or concaved or recessed or otherwise deformed to accommodate the shape of the head of the tab. That is, the head of the tab can have a shape that at defines a portion of the cross-sectional profile of the aperture where such portion causes the aperture to be not perfectly circular, which can be described as the aperture being eclipsed with the tab. The aperture can have a cross-sectional profile that is partially circular, where a circular part is defined by a wall of the main body and a non-circular part or eclipsed part is defined by a wall of the head of the tab.

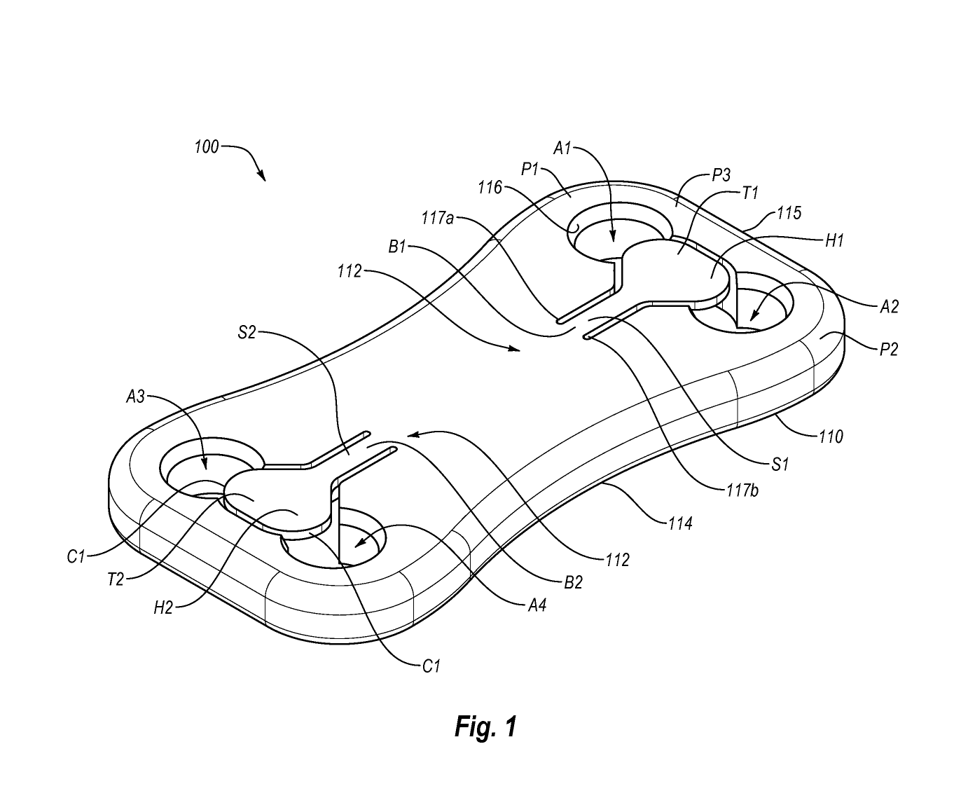

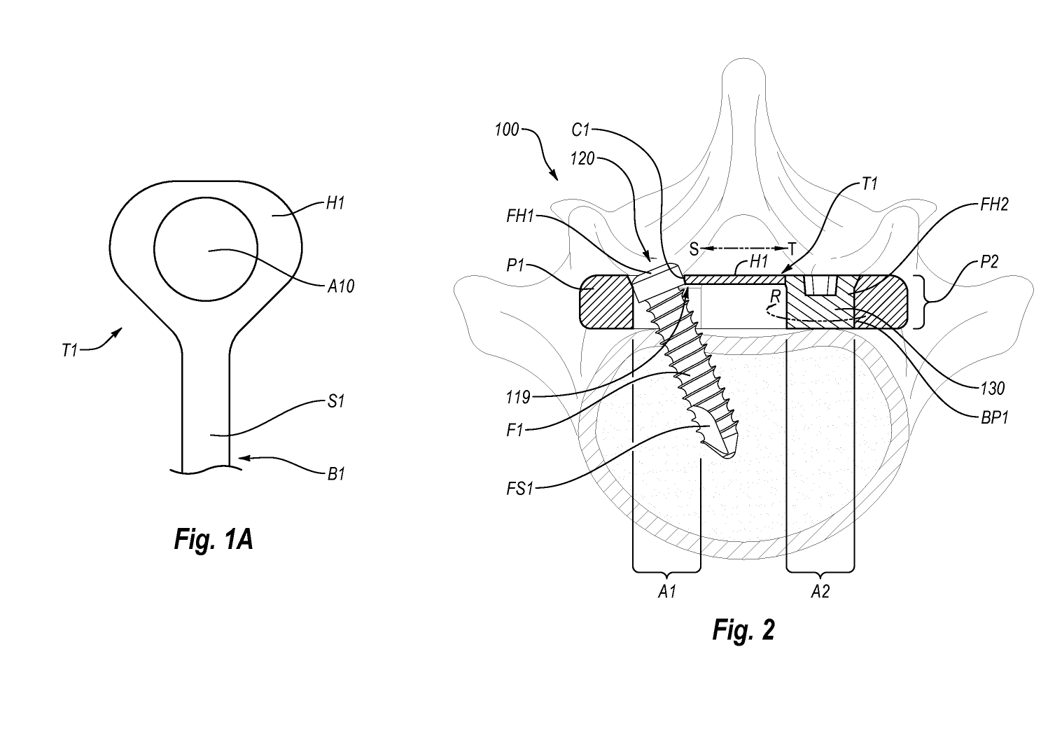

The figures illustrate general embodiments of a bone plate implant of the invention. FIG. 1 shows an implant 100 with a unitary body 110 configured according to the principles of the invention. Generally, the implant 100 can be considered a bone plate 100. The bone plate 100 can include one or more apertures, such as four apertures A1, A2, A3 and A4, formed into a main body portion 112. A tab T1 can be positioned intermediate apertures A1 and A2, and a tab T2 can be positioned intermediate apertures A3 and A4. The tabs T1, T2 may also be part of the body 110 and can be contoured or shaped to lay in the plane of the main body portion 112. Tab T1 can include a base B1 that is coupled with the main body portion 112, a stem S1 coupled to the base B1, and a head H1 coupled to the stem S1 opposite of the base B1. Tab T2 can include a base B2 that is coupled with the main body portion 112, a stem S2 coupled to the base B2, and a head H2 coupled to the stem S2 opposite of the base B2. The apertures A1 and A2 can be opposite of the apertures A3 and A4 so that the head H1 of tab T1 points away from the head H2 of tab T2.

The apertures A1 and A2 can each be a single aperture or hole or conduit with a partially or substantially circular cross-sectional shape, which shape can range from circular to crescent or eclipsed or any shape therebetween. Also, the apertures A1 and A2 can be formed from a single larger aperture receiving the tab T1 therein so as to form two apertures (e.g., A1 and A2) due to the tab T1 being located in the larger aperture. That is, the larger aperture can be divided into the apertures A1 and A2 by the tab T1. When tab T1 is bent upward or downward with respect to the plane of the body 110, apertures A1 and A2 can form a single larger aperture along with the space vacated by tab T1. Similarly, the apertures A3 and A4 can each be a single aperture or hole or conduit, or be formed into two apertures due to the tab T2 being located within a larger aperture. When tab T2 is bent upward or downward with respect to the plane of the body 110, apertures A3 and A4 can form a single larger aperture along with the space vacated by tab T2. The implant 100 can have any number of apertures with tabs and apertures A1-A4 and tabs T1-T2 can be positioned anywhere along and within the implant, and the apertures A1-A4 and tabs T1-T2 may have any orientation with respect to the body of the implant 100. While FIG. 1 shows an embodiment of a bone plate implant 100, the shapes and dimensions of the various described components thereof can be modified or altered.

The tab T1 can be thinner than the main body portion 112 of the implant 100. As such, the stem S1 and/or head H1 of the tab can be thinner than the main body portion 112, or portions P1 or P2, and therefore the tab T1 can be more deformable than the main body portion 112 or portions P1 or P2 of the implant 100. Portions P1 and P2 are lateral or more toward a side edge 114 of the implant 100. The shape of the portions P1 and P2 can define the shape of the apertures A1 and A2, respectively. As show, portions P1 and P2 include a wall 116 that defines the apertures A1 and A2. Wall 116 can extend from point 117a to point 117b, and thereby define the apertures A1 and A2 as well as the tab T1. Tab T1 can be cut from the main body portion 112 so as to form the corresponding portion of the wall 116, and apertures A1 and A2 can be made to form the corresponding portion of the wall 116. Portion P3 can be located between the Tab T1 and end edge 115 and include a portion of wall 116 and thereby define a portion of the free head H1 of the tab T1. As such, portion P3 is between portions P1 and P2, where the interface therebetween can be defined by the lateral sides of the head H1 of the tab T1. These portions and walls can also be applied with apertures A3 and A4 and tab T2.

In one embodiment, the shapes of the apertures A1 and A2 and tab T1 can be defined by a single cutout from the body 110 of the implant 100. That is, a single cut can be made from point 117a to point 117b so as to form a single opening or hole through the body that has the shape shown for the apertures A1 and A2, extending from the apertures A1 and A2 into the main body portion 112 so as to define the stem S1 of the tab T1, and extending between the apertures A1 and A2 so as to define the head H1 of the tab T1. Accordingly, a single complex-shaped hole or hole and series of slits can provide the shape of the apertures A1 and A2 and the stem S1 and head H1 of the tab T1. One or more cuts into the main body portion 112 can form the stem S1 and head H1 of the tab T1 where a hole connected to the one or more cuts can define an aperture. Two holes connected to the one or more cuts can define apertures A1 and A2 or A3 and A4 as shown in FIG. 1.

An embodiment of the invention provides a bone plate implant configured for attachment to a vertebra. The implant can include at least a unitary body with an aperture and a tab. The implant can be shaped as a circle, oval, triangle, square, rectangle, other polygon, “C,” “E,” “F” “H,” “I”, “J,” “L,” “T,” U,” “V,” “X,” “Y” or other alpha or numeric shape.

In one embodiment, the bone plate implant includes at least aperture configured for receiving a fastener therethrough so as to fasten the implant to bone. The aperture can be located on any portion of the body, such as in a main body region or arm body region. The aperture can be at least partially defined or covered by a tab. As such, the shape of the cross-sectional profile of the aperture can be partially defined by the main body portion of the implant and partially by the tab. The main body portion may define a circular cross-sectional profile of the aperture which can be eclipsed with a portion of the tab so that the cross-sectional profile of the aperture appears to be in the shape of an eclipse. This is similar to the moon (e.g., tab) eclipsing the sun (e.g., aperture). The shape of the eclipsed cross-sectional profile of the aperture can be varied depending on the shape of the head of the tab and the dimension of the side lobes of the head. Generally, when the head of the tab protrudes into or over the aperture, the cross-sectional profile of the aperture can be considered to be eclipsed. In a relaxed position the eclipse can be smaller than when the tab is bent into the aperture. In the relaxed position, the eclipse can be about 0% to 75%, 0.01% to 70%, 0.1% to 60%, 1% to 50%, 5% to 40%, 10% to 30%, or 15% to 20% of a circular aperture. Generally, the strained position that causes greater eclipse of the aperture by the head of the tab can cause higher percentages of eclipse, such as 1, 2, 3, 4, 5 or other integer times the size of the relaxed eclipse. However, the aperture may also be a polygon that is eclipsed by a circular or polygon-shaped tab head. In fact, the aperture can be any shape that can receive a fastener and interact with the tab as described herein.

The tab can include a base coupled to or integrated with a main body portion of the body, a stem extending from the base, and a free head portion opposite of the base. The free head portion can freely move with respect to the main body portion by moving the base and/or stem with respect to the main body portion, which movement can also be with respect to one or more apertures. The tab can be flexibly resilient and capable of moving laterally or orthogonally or any direction with respect to the aperture. In one aspect, the base of tab provides a pivot point and the stem allows for the head to be moved in an arcuate movement pattern with respect to the main body portion or apertures. The head of the tab can be flexibly displaced by a fastener, such as a bone screw, when the fastener is inserted through the aperture and into a vertebra. The displacement of the head can be arcuate with respect to the pivot point at the base of the stem. Accordingly, the body of the bone plate implant can include at least one tab that is pivotally or flexibly or otherwise attached to a main body portion of the body so that the tab can pivot, flex, bend or otherwise move with respect to the main body portion of the body, where the base of the tab can provide the pivot point, flex point, or bending point. The free head of the tab can be elastically displaced (e.g., arcuately displaced) from the aperture by a bone screw and the tab can thereby be resilient and press against the screw so as to elastically prevent the screw from displacing from the aperture. The tab can have various shapes and configurations with respect to the main body and/or the apertures. The tab can be configured with at least one portion of the tab (e.g., free head) that is adjacent to the aperture, defines a portion of the aperture, or extended into the aperture. The free head of the tab can be being wider than the base and/or stem. The wider free head portion of the tab may be shaped as wings or lobes on a stem of the tab that is attached to the body; however, any suitable shape, whether polygon or rounded can be used.

In one embodiment, the body of the bone plate implant can be attached to a second body. The second body can be a different implant or another body portion of the bone plate implant. For example, another implant can be any of the implants described in U.S. patent application Ser. No. 13/370,925, U.S. Provisional Application Ser. No. 61/554,589, U.S. Provisional Application Ser. No. 61/554,600, or U.S. Provisional Application Ser. No. 61/554,616, which are incorporated herein by specific reference in their entirety, especially figures and portions thereof describing implants. Also, the body of the bone plate implant can include a first body and a second body, where the first body is attached to the second body, such as when two arms are attached or a main body portion is attached to an arm. The attachments can be by integration, coupling, fastening, adhering, or the like. The attachments may or may not be via a fastener through apertures such as those described herein. The attachments can be fixed, pivotally attached, flexibly attached, rotatably attached, or the like to at least a second body.

FIG. 1 shows that tab T1 and tab T2 are integrated with the body through base B1 and base B2. Base B1 and base B2 are normally narrower than the stems S1 and S2 and/or heads H1 and H2. tab T1 and tab T2. Base B1 and base B2 can be coupled or integrated with the main body portion 112 of the implant 100.

The implant can be prepared from one or more materials that are biocompatible. Examples of materials can include polymers, ceramics, composites, metals, alloys, hybrid materials, and combinations thereof, which can be biostable or biodegradable or resorbable. The materials of the tabs and main body can be the same or different. When the same, the tab can be cut out from the material of the main body. When different, the tab can be coupled to the main body through a coupling member, where the coupling member can provide for the flexibility or bendability or pivot point of the tab with respect to the main body. The coupling can be a member, such as polymers, ceramics, composites, metals, alloys, hybrid materials, and combinations thereof, or an adhesive or fastener member.

FIG. 1A shows an alternative embodiment of a tab T1 that includes an aperture A10 in the head H1. The aperture A10 in the head H1 can be dimensioned or otherwise configured for receiving a fastener, such as a bone screw. The tab T1 can be arranged with apertures in a main body as described herein. The head H1 can be coupled or integrated with the stem S1, which in turn is coupled or integrated with a base B1 that is coupled or integrated with the main body portion 112 of the body 110 of the bone plate 100. When the head H1 includes the aperture A10, the bone plate 110 can be fastened to bone by one or both of apertures A1 and A2 receiving a fastener therethrough, and then aperture A10 receiving a fastener. Alternately, aperture A10 can receive the faster before one or both of apertures A1 and A2. In another alternative, only aperture A10 can receive a faster with respect to apertures A1 and A2 that either don't receive a fastener, or receive a fastener once the bone plate 100 has be positioned by moving the main body portion 112 by bending or flexing the base B1. In one aspect, a fastener can be installed into the aperture A10 of the tab T1 before, during, or preferably after fasteners are installed into the apertures A1 and/or A2 adjacent to the tab T1, where the tab T1 can still function to retain the fasteners in the apertures A1 and/or A2.

The implant of FIG. 1 can be installed onto a bone surface by insertion of fasteners into the apertures and into the bone. The fasteners can be inserted into one or more of the apertures. The tab can be displaced, such as by rotation or pivoting in or out from the plane of the implant body or along the plane of implant body, during insertion of a fastener into the aperture. The tab can then be replaced so as to contact the faster, friction couple the fastener, or cover the fastener or otherwise facilitate retention of the fastener into the aperture. The tab can then be moved away from the fastener, and the fastener withdrawn when or if the implant is removed from the bone surface.

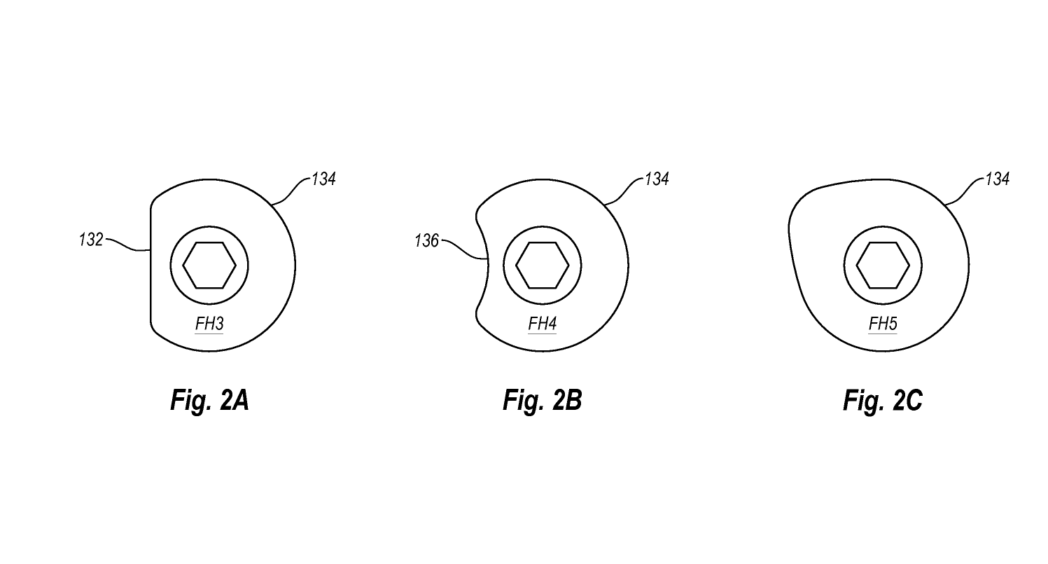

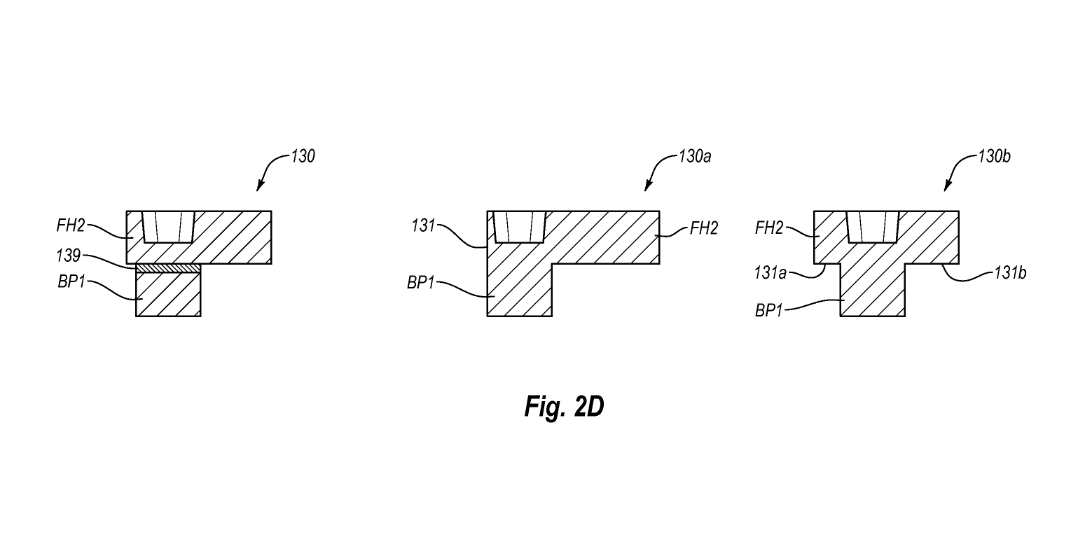

FIG. 2 illustrates a side view of an embodiment of a bone plate fastening system 120. The fastening system 120 can include the bone plate implant, such as implant 100 of FIG. 1A, and at least one fastener, such as fastener F1, having a fastener head FH1. Optionally, the fastening system can include or use a cam 130 with a rotatable fastener head FH2 to cam the tab T1. As shown, the implant 100 includes a screw fastener F1 inserted through aperture A1 and a cam 130 with a rotatable fastener head FH2 in aperture A2. The cam 130 can include a base portion BP1 that is integrated with, coupled to, or rotatably coupled to the rotatable fastener head FH2. As such, rotation of the rotatable fastener head FH2 may rotate only the fastener head FH2 or the entire cam 130 with respect to the aperture A2.

The implant 100 can be configured as any implant as described herein having the apertures (e.g., A1-A4) and tabs (e.g., T1 and T2). The screw fastener F1 can be any type of fastener that can fasten the implant 100 to a bone, and can include a fastener head FH1 that is adapted to receive a fastener device, such as a screw driver, wrench, bolt driver, ratchet, or the like. For example, the fastener head FH1 and rotatable fastener head FH2, and any other fastener head described herein can have protrusions and/or recesses adapted to receive a fastener device, where philips, hex, and torx screw recesses can be provided as examples.

The cam 130 having the rotatable fastener head FH2 can be a fastener that fastens to bone or it can be a member that is received into the aperture A2 in the fastener receiving portion opposite of the aperture A1 that receives the screw fastener F1. That is, the cam 130 having the rotatable fastener head FH2 may or may not be attached to a threaded screw portion, shank, or other fastener member. As such, the cam 130 with the rotatable fastener head FH2 may be rotatably coupled with aperture A2 so that rotation thereof can facilitate movement of the free head H1 of the tab T1. For example, the rotatable fastener head FH2 can be similar to a screw head without the threaded portion. The base portion BP1 of the cam 130 can have grooves, or protrusions, threading, or other coupling features that mate with or are received with corresponding coupling features of the Aperture A2. Also, the base portion BP1 of the cam 130 may be integrated with the cam 130 such that the fastener head FH2 rotates around the base portion BP 1.

When the base portion BP1 of the cam 130 is rotatable with respect to the fastener head FH1, the base portion BP1 can be locked, fixed coupled, fastened, adhered, or integrated with the aperture A2 so that rotation of the fastener head FH2 only rotates the fastener head FH2, but not the base portion BP1. As such, aperture A2 can be omitted and the implant can include the cam 130 integrated therewith by the base portion 130 being coupled or integrated with the implant so that the fastener head FH2 is rotatable with respect to the base portion 130 and/or implant to perform the cam action.

In one example, the cam 130 and/or fastener head FH2 can be a member with or without a screw portion. If having a screw portion, the length of the screw shank can be long enough to pass through the implant 100 and into bone or the screw portion can be short so as to only fit into and engage with the walls of the implant that define the aperture A2. If devoid of a shank, the cam 130 and/or fastener head FH2 can include a bolt portion BP1 (e.g., base portion BP1) that is dimensioned in length to fit neatly into the aperture A2 without contacting the bone.

The fastener head FH2 can be round or it can be asymmetrical or configured as a cam. The cam 130 and/or bolt portion BP1 can be cam shaped. The cam 130 and/or rotatable fastener head FH2 can be received into the aperture A2 without pushing on the tab T1, and then rotated so that the cam 130 and/or fastener head FH2 then pushes against the tab T1. As such, the rotatable fastener head FH2 can function as a cam. Some representative cross-sectional profiles of the rotatable head FH2 can be seen as: FH3 in FIG. 2A which has a flat left edge 132 and round edge 134; FH4 in FIG. 2B which has a concave edge 135 and round edge 134; FH5 in FIG. 2C, which has an oblong cross-sectional profile. However, other shapes of the rotatable fastener head FH2 that can provide a cam feature can be used. These cross-sectional profiles can also be applied to the cam 130 and/or bolt portion BP1.

FIG. 2D illustrates: a cam 130 with a fastener head FH1 rotatably mounted to the bolt portion 130a through a rotation member 139; a cam 130a with a fastener head FH2 asymmetrically fixed to the bolt portion BP1 with a common edge 131; and cam 130b with a fastener head asymmetrically fixed to the bolt portion BP1 with a short side 131a and a long side 131b to provide the asymmetry. Any of these may have the fastener head FH1 rotatably or fixed with respect to the bolt portion BP1.

FIG. 2 also illustrates an embodiment of mounting the implant 100 to a vertebra. As shown, the fastener F1 can be mounted through the implant 100 and into the vertebra by being inserted through aperture A1. The fastener F1 can be inserted so that the fastener head FH1 is flush or countersunk into the implant 100, or as shown the fastener F1 can be inserted so that the fastener head FH1 can protrude from the aperture A1. The protrusion of fastener head FH1 can be beneficial in some instances when there isn't an ability to insert the fastener F1 further into the implant 100 and/or vertebra.

Tab T1 is shown intermediate apertures A1 and A2 of the implant 100, such as in FIG. 1. Tab T1 is thinner and therefore more deformable than portion P1 or P2 of the implant. The elasticity or of tab T1 (e.g., elasticity of stem S1) allows tab T1 to move in the direction of arrows S and T when a fastener F1 is inserted in aperture A1. Rotatable fastener head FH2 is configured to accept a screw driver and rotate in the clockwise or counterclockwise direction of arrow R. Rotating fastener head FH2 thereby moves tab T1 in the direction of arrows S and/or T to move portion C 1 of the head H1 of tab T1 to interfere with fastener F1. Fastener F1 is preferably inserted “flush” that is completely turned into the aperture A1 so that the fastener head FH1 is flush with the surface of portion P1 of the body 112 of the implant 100.

One advantage of the invention is that fastener F1 can be inserted “proud” or at least partially turned out with respect to the surface of portion P1. Rotating fastener head FH2 functions as a cam to push the free head H1 of the tab T1 so as to interfere with fastener head FH1. A recess 119 in the fastener F1, such as in fastener head FH1 of fastener shank FS1, can be optionally provided and configured such that the portion C1 of the head H1 of tab T1 can fit into the recess 119 to prevent fastener F1 from “backing out” of the aperture A1 with respect to the surface of portion P1 in the direction opposite that used for insertion. The recess 119 can be a slit or groove, and can extend circumferentially, or a portion around the circumference of the fastener shank FS1 or fastener head FH1 or connection portion therebetween. Portion C1 of the head H1 can be a surface or projection that is received into a slit or groove recess 119 so as to receive the tab T1 to prevent rotation of fastener F1.

In one embodiment, the tab T1 can be coupled with the fastener F1, such as at fastener head FH1 or fastener shank FS1. As such, the head H1 of elastic tab T1 can be fused, welded, adhered, glued, cold welded, brazed or otherwise coupled with the fastener head FH1 or fastener shank FS1. The coupling of tab T1 and the fastener F1 can be by friction coupling. The coupling of head H1 and the fastener F1 can be accomplished after or by rotating the fastener head FH2 of the cam 130. For example, the fastener head FH2 can be shaped as a partial sphere as shown, such as in FIGS. 2A-2D. The fastener head FH2 preferably is cam shaped so as to be wider in at least one dimension so as to cam when rotated. The fastener head FH2 can have any shape and dimension that functions as a cam to push the head H1 of the tab T1 into the fastener F1. The ability of tab T1 to interfere with fastener F1 by rotating head FH2 to flexibly (elastically) deform tab T1 provides a reversible mechanism for locking fastener F1 in implant 100. For example, the fastener F1 can be angularly placed as shown within aperture A12 of implant 100, and fastener head FH1 does not need to nest entirely within aperture A1. Rotating fastener head FH2 is shown fully nested within aperture A2 in implant 100, but can be angled, oblique or pivoting. The locking mechanism 120 described herein can be on an implant 100 configured to attach to an intervertebral implant, such as an expandable device implant, or other vertebral implant when screws are used to fasten the implant 120 when configured as a bone plate along a vertebra. The vertebral implant can be any of the implants described in U.S. patent application Ser. No. 13/370,925.

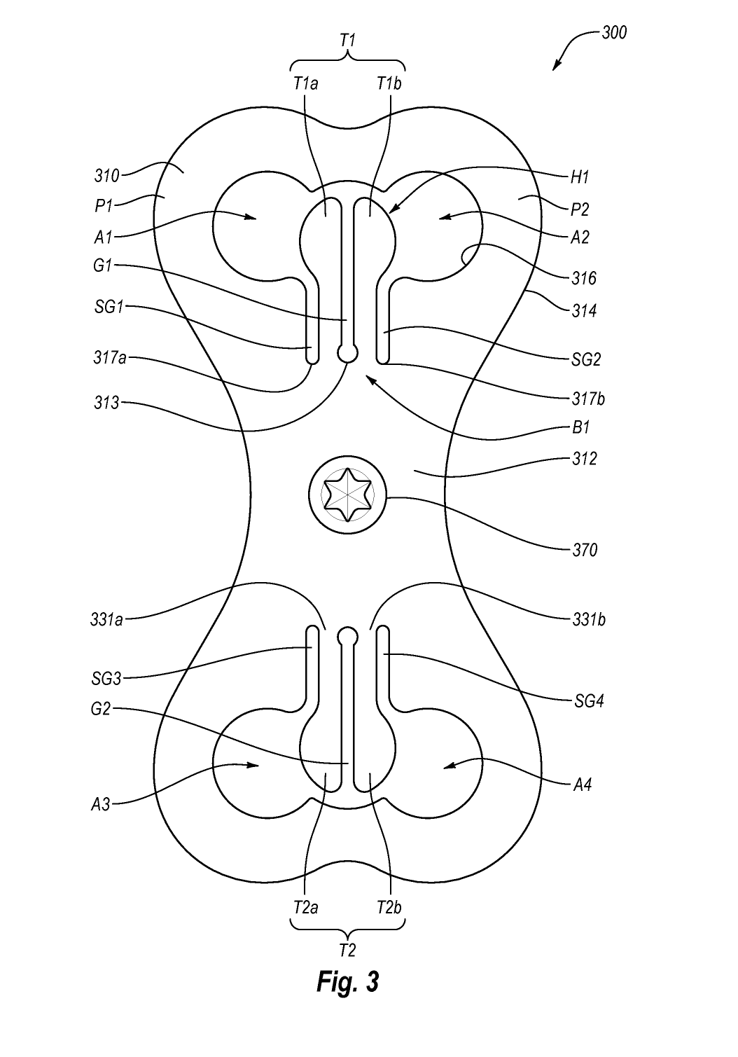

FIGS. 3-5 show different embodiments of a bone plate having the apertures and tabs as described in connection to FIGS. 1 and 2. Any of the features shown in one figure can be combined or applied with the features of another figure.

FIG. 3 shows an implant 300 having slotted grooves SG1, SG2 that are formed in the body 310 of the implant 300 so as to be adjacent to tabs T1 and T2, respectively. The slotted grooves SG1, SG2 can be cut partially into the main body portion 312 as recesses or all the way through the main body portion 312. As shown, the aperture A1 is connected to the slotted groove SG1 on one side of tab T1, and aperture A2 is connected to the slotted groove SG2 on the other side of tab T2. On the other end, the aperture A3 is connected to the slotted groove SG3 on one side of tab T2, and aperture A4 is connected to the slotted groove SG4 on the other side of tab T2. Tab T1 includes a center groove G1 that extends from the head H1 to base point 313 in the base B1, and thereby tab T1 is split into tab flaps T1a, T1b as illustrated. The center groove G1 can be cut partially into the tab T1as a recess or all the way through the tab T1. Tab T2 also has a corresponding center groove G2 that forms tab flaps T2a, T2b. The groove G1 allows for the tab flaps T1a and T1b to move with respect to each other, and thereby tab flap T1a can move without tab flap T1b moving. As such, a fastener can be inserted into aperture A1 so as to move tab flap T1a without moving tab flap T1b, where tab flap T1a can engage the fastener as described herein, such as a friction engagement that inhibits the fastener from being withdrawn from aperture A1. Similarly, tab flap T1b can be engaged by a fastener in aperture A2 without disturbing or moving tab flap T1a. Similarly, fasteners can be inserted in apertures A3 and A4 to engage tab flaps T2a and T2b, respectively, without disturbing the other tab flap. As such, the tab T1 can engage a fastener in aperture A1 without disturbing or covering aperture A2, or vice versa, and the tab T2 can engage a fastener in aperture A3 without disturbing or covering aperture A4, or vice versa.

The tab flaps T1a and T1b can be substantially thinner and therefore more deformable than portion P1 or P2 of the implant 300. Portions P1 and P2 are lateral or more toward a side edge 314 of the implant 300. The shape of the portions P1 and P2 can define the shape of the apertures A1 and A2, respectively. As show, portions P1 and P2 include a wall 316 that defines the apertures A1 and A2. Wall 316 can extend from point 317a to point 317b, and thereby define the apertures A1 and A2 as well as the tab flaps T1a and T1b. Tab flaps T1a and T1b can be cut from the body 310 so as to form the corresponding portion of the wall 316, and apertures A1 and A2 can be made to form the corresponding portion of the wall 316. Also, wall 316 can extend around both tab flaps T1a and T1b and into the groove G1 and around point 313. Point 313 can be rounded as shown so as to improve bendability of the tab flaps T1a and T1b. All of the apertures and tabs and/or tab flaps can be similarly configured.

The tab flaps T1a and T1b can be the same material as the main body, such as portion P1 or P2, but can be of different material. Any method (e.g., molding or laser shaping) can be used to form the apertures A1 and A2, tab flaps T1a and T1b, slotted groove SG1 and SG2, and groove G1 in the body 310 of the implant 310. The method of formation can provide the slotted groove SG1 and SG2 so that the tab flaps T1a and T1b have elasticity, bendability, or flexibility to function as described herein to bend with respect to the body 310.

The tabs or tab flaps can have narrow bases. The narrow bases 331a and 331b of tab flaps T2a and T2b allow pivitol or bendable (e.g., arcuate pivotal or bendable) movement of tab T2 when a fastener, such as a screw (not shown), is inserted in the plate 300 through aperture A3 and/or aperture A4. This can also be applied to tab flaps T1a and T1b as well as larger tabs T1 and T2.

Additionally, FIG. 3 shows a fastener 370 that is received through aperture (not shown) in the main portion 312 of the body 310 of the implant 300. While not shown, the aperture receiving the fastener 370 may also include a tab as described herein. This fastener 370 is adapted to be received into an intervertebral implant, such as an expandable implant, spinner implant, linked implant, or other implant such as shown in the incorporated references. The fastener 370 can be adapted so that rotation thereof can operate the intervertebral implant. Accordingly, the fastener 370, which may be referred to as an intervertebral implant fastener 370, and/or the intervertebral implant can be included in a kit or system of the present invention. The system can include the intervertebral implant operably coupled to the fastener 370 such that rotation of the fastener 370 operates, articulates, or otherwise manipulates the intervertebral implant. In one example, the intervertebral spacer operably coupled to the fastener 370 and bone plate 300 can be inserted between adjacent vertebra, such as within the disc, and then the fastener can be rotated so as to operate the intervertebral spacer to expand or spin so as to maintain or increase the distance between the adjacent vertebras or cause internal traction to separate the adjacent vertebras. After the intervertebral implant is set, fasteners can be inserted into the apertures A1-A4 and into the adjacent vertebras (e.g., A1-A2 in one vertebra and A3-A4 into the other vertebra) to fix the bone plate 300 to the adjacent vertebras. The fastener 370 may or may not be disengaged from the intervertebral implant once it is set between the adjacent vertebras.

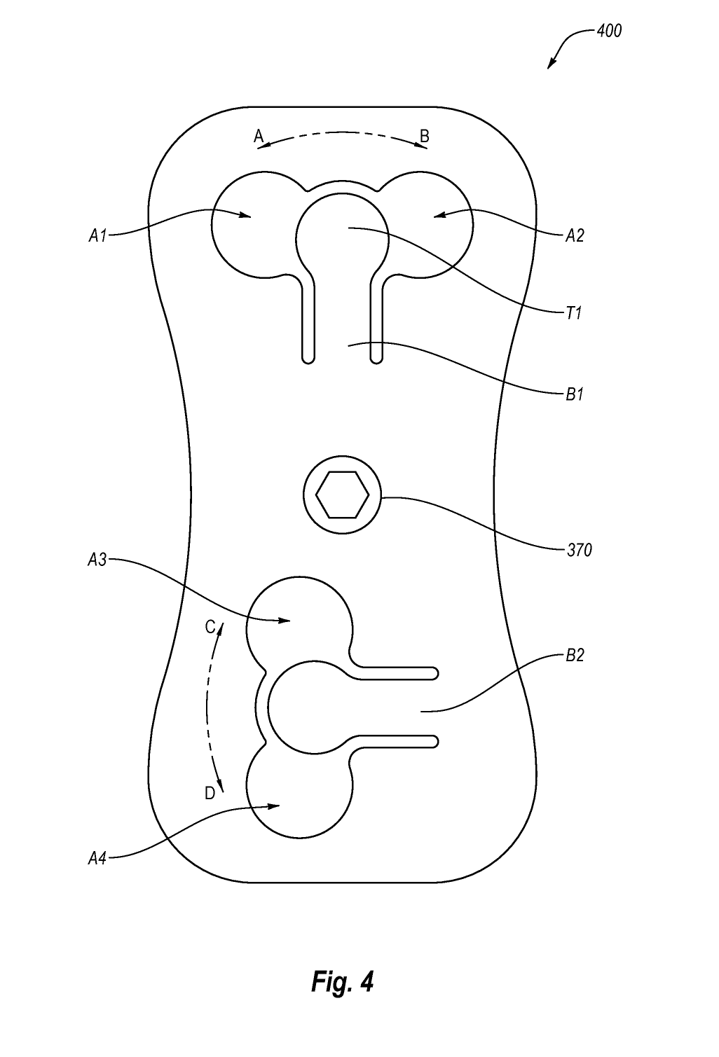

FIG. 4 illustrates the bendability of a tab T1 with respect to apertures A1 and A2 and bendability of tab T2 with respect to apertures A3 and A4. Also, tab 2 is shown to be at an angle from tab T1, such as being orthogonal, but it can at any angle be from about 0-180 degrees, such as about 10-170 degrees, about 20-160 degrees, about 30-150 degrees, about 40-140 degrees, about 50-130 degrees, about 60 to 120 degrees, about 70 to 110 degrees, about 80 to 100 degrees, or about 90 degrees as illustrated.

The implants described herein can be implanted as normally practiced, with the exception that the tabs operate as friction couplings or covering flaps to inhibit the fasteners from being withdrawn from the apertures. As is normally practiced, a surgeon typically inserts a fastener “one at a time” within aperture A3 and aperture A4, allowing tab T2a to move “back and forth” or otherwise flex or bend in the arcuate direction of arrow A and B, or Tab T2 to move in the direction of arrows C and D. When tabs T1 and T2 move back and forth, apertures A1 and A2 are uncovered and covered, allowing fasteners to be inserted through aperture A1 and then locked or blocked from backing out of aperture A1 and A2 when the fastener is inserted through aperture A2. The tabs can provide friction coupling with the fasteners, where the friction coupling inhibits rotation or withdrawal of the fasteners from the apertures. Insertion of the fastener into the second aperture A2 can push the tab back toward the fastener in the first aperture A1, thereby creating friction against both the fasteners in both apertures A1 and A2. Also, fasteners (e.g., screws) can be inserted simultaneously in the apertures A1 and A2 to strain or deform the tab T1. To facilitate simultaneous application of fasteners, the tab T1 can have the groove G1 shown in FIG. 3 so that tab T1 is divided into tab flaps T1a and T1b. Both tab flaps T1a and T1b can bend toward each other for simultaneous insertion of fasteners through apertures A1 and A2. However, simultaneous fastener insertion through apertures A1 and A2 may be obtained with an implant with a unitary tab. Once fasteners are inserted in apertures A1 and/or A2, tab T1 can normally recoil or elastically move in the arcuate direction of arrows A or B to cover and/or interfere and/or friction couple with the fasteners. The tab covering or interfering or friction coupling with the fasteners functions to prevent the fasteners or screws from backing out or moving in the direction opposite the direction of insertion that is usually normal to the body of the implant (e.g., aligned with the aperture receiving the screw). The tab T1 can also be laterally compressed between fasteners in apertures A1 and A2.

In one embodiment, the tabs can be lobed, oblong, or “omega” shaped, that is, wider adjacent apertures A1 and A2 than at the stem or base portion coupled with the body. The tabs can be any shape and dimension such as square, round, swirl, star, hexagonal, hooked, or the like, as long as the tab functions to at least partially interfere, eclipse, cover, or friction fit with a fastener. In one aspect, the tab can include a head that is round and configured to arcuately displace with respect to the base of the stem or body of the implant when a fastener is inserted in aperture A1. The stem or base can be linear and of uniform shape and dimension (shown), or the stem or base can have a variable shape and dimension such as curved, acruate, and/or tapered with uniform or variable stiffness. For example, the stem can be wider at the head than the base portion, or the stem can be narrower at the head than the base portion. Also, the stem can be of any shape that can connect the head to the body of the implant.

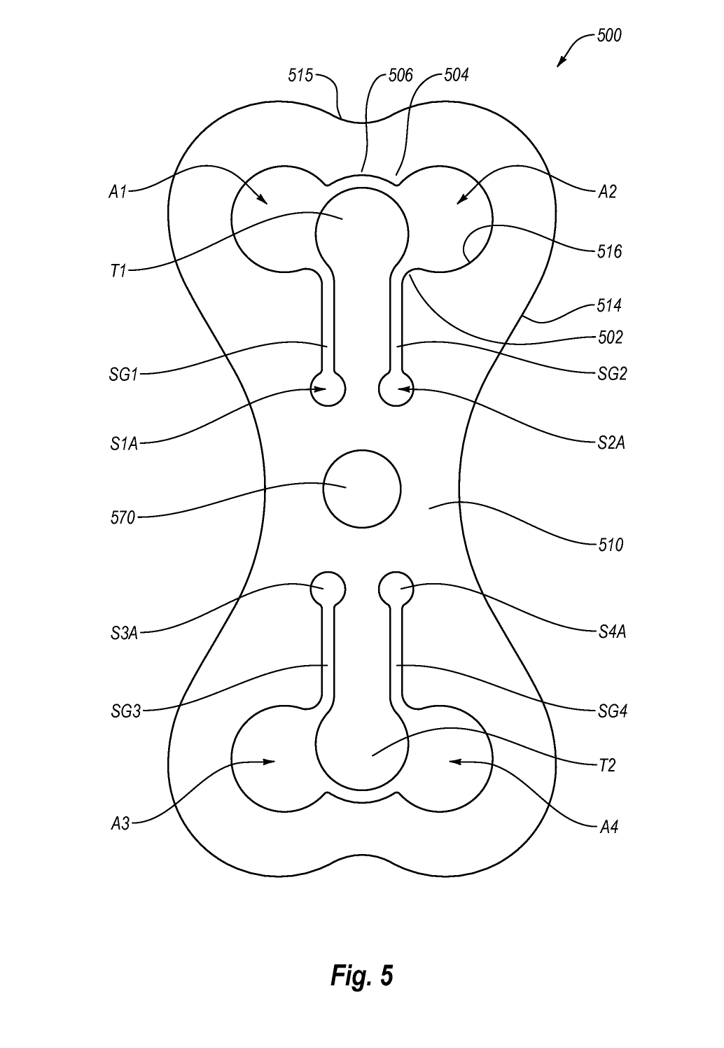

FIG. 5 shows in implant 500 having a body 510 that is similar to the implant 100 of FIG. 1. However, the side 514 and end 515 are more concave or rounded. This shows the implant can have either straight or rounded edges. Also, the tabs T1 and T2 are more rounded with less side lobes compared to FIG. 1. The slotted grooves SG1, SG2, SG3, and SG4 (e.g., similar to FIG. 3) can terminate to slotted apertures S1A, S2A, S3A, and S4A, respectively. The slotted groves SG1, SG2, SG3, and SG4 and/or slotted apertures S1A, S2A, S3A, and S4A can be recesses in the body 510, or cuts or slits that extend all the way through the body. The wall 516 can also be rounded such as at points 502, 504, 506, and at any other location with respect to the apertures, tabs, slotted grooves, or slotted apertures.

The implant shown in the figures can include a height, a length, and a width, which can be varied as desired. Also, the tabs can each have a height, a length, and a width, that is normally less than height, length, and width w of the implant. The shape and dimension of the tab and apertures, grooves, and slotted grooves can vary as desired.

In one embodiment, an implant for attachment to a vertebra can include a unitary body with an aperture. The aperture can be at least partially covered by a flexible or movable tab. The tab can be flexibly or movably displaced by a fastener when the fastener is inserted through one of the apertures adjacent to the tab and into a vertebra. The bone plate implant having the body, apertures, tabs, slotted grooves, tab grooves, slotted apertures, or other features as described herein can have various features, such as including, but not limited to the following: said tab is pivotally attached to the implant body; the implant body has a thickness greater than the tab; the tab is arcuately displaced from an aperture by a screw and elastically prevents the screw from displacing from the implant body or aperture thereof; the body is pivotally attached to at least a second body (e.g., second body of same implant or of a different implant, here the bone plate can hold an implant in place upon implantation); and/or the tab is configured with at least one portion of the tab, adjacent an aperture, wider than the base of said tab.

In one embodiment, the implant can include a body having a length, width, and thin thickness. The body can include one or more apertures extending from a front surface to a back surface. Each aperture can have two or more fastener receiving portions. A tab for each of the apertures can be positioned between each of the two or more fastener receiving portions of the apertures. The tab can be integrated or coupled with the body so as to be flexible with respect thereto and bendable or rotatable or capable of pivoting in an arcuate manner with respect to the two or more fastener receiving portions of the apertures. The implant can also be configured in accordance with one or more of the following: the tab has a wide head and a thin stem, the thin stem being integrated or coupled with the body; an aperture on each end of the length of the body; the tab includes a groove such that the tab has two separate tab flaps or tab portions, each tab flap having a wider head and thinner stem; the tab is defined by a slits or slotted grooves in the body on each side of the tab; each aperture and tab are arranged in opposite orientations; at least two aperture and tab pairs are arranged so as to be angled with respect to each other; or the apertures and tabs are rounded without sharp corners.

In one embodiment, a kit can include the implant having the apertures and tabs as described herein along with one or more fasteners and/or one or more cams, the fasteners and/or cams being adapted to be received into the apertures. Optionally, the kit can include one or more cams or one or more fasteners, or both cams and fasteners as described herein. In one option, the implant includes the cam with a rotatable fastener head mounted therein. In one aspect, the implant described herein can be provided as a kit with one or more fasteners (e.g., F1) and/or one or more cams (e.g., cam 130) and/or rotatable fastener heads (e.g., FH2). The implant, fasteners, cams, and/or rotatable fastener heads can be prepared from the same or different biocompatible materials, such as metals, ceramics, polymers, or others.

In one embodiment, a method of mounting an implant to a vertebra can include: providing an implant having the apertures and tabs as described herein; inserting a fastener through the fastener receiving portion of an aperture so as to move the tab toward the corresponding fastener receiving portion of an adjacent aperture; and screwing the fastener into the vertebra. The method can also include pivoting the tab before, during, and or after the insertion of the fastener, where the pivoting can be away from the fastener or toward it. The method can include inserting a cam with a rotatable fastener head through the corresponding fastener receiving portion of the adjacent aperture that corresponds with the fastener receiving portion or aperture having the fastener; and rotating the rotatable fastener head so as to pivot or cam the tab toward or against or over the fastener.

In one embodiment, the implant, tab, or screw of the implant can have one or more teeth, protuberances and or recesses to interlock or reversibly interlock with the implant, tab and/or screw as desired. That is, the surfaces thereof can include cooperative features that can be received into each other and interlocked. The surfaces can be configured as any of the surface of any of the implants in any of the incorporated documents.

In one embodiment, the surface of the implant can include recesses, such as a bone-contacting surface having recesses that can receive bone growth therein. The recesses can be narrower at the opening than the base, or wider at the opening that the base, or have a uniform cross-sectional profile. These bone-receiving recesses can function to lock the implant to bone when bone grows therein. Also, the recesses can be filed with polymers or other compositions that can contain bone growth factors or other active agents to promote bone growth into the bone-receiving recesses.

In one embodiment, a locking plate mechanism is provided for preventing a fastener, such as a screw, from backing out of a bone plate once attached to a vertebra. The bone plate can include a tab that plastically or elastically deforms in response to pressure from a screw in at least one direction and resists deformation in at least one direction once the screw is inserted into the bone plate. The bone plate can be a unitary structure of variable thickness or part of an assembly including an intervertebral body spacer and/or hinged plate and/or other intervertebral implant, where the bone plate can be rotated along an axis to expand a core within the intervertebral body spacer. For example, the intervertebral implant can be as described in U.S. Provisional Application Ser. Nos. 61/554,600 and 61/554,616 and U.S. patent application Ser. No. 13/370,925, which are incorporated herein. The bone plate can be configured with at least one elastically deformable tab to fasten and lock the device to a vertebra. Fixing the bone plate to a vertebra can prevent the bone plate or implant coupled thereto from moving. When bone plate is coupled to an implant, once the implant device is rotated, expanded, separated, joined, articulated, pivoted, or otherwise manipulated, the bone plate can be attached to bone to inhibit the implant from being further rotated, expanded, separated, joined, articulated, pivoted, or otherwise manipulated. After implantation, an instrument (e.g., flat head screwdriver) can be inserted into the bone plate in order to reversibly deform the tab and uncover the fastener from the bone plate so that the same instrument or a fastener driver can rotate the fastener, thereby allowing the implant to be moved rotated, expanded, separated, joined, articulated, pivoted, or otherwise manipulated.

In one embodiment, the implant can be implanted by a method of implantation that includes: obtaining an implant having one or more apertures and one or more tabs associated with each aperture; obtaining one or more fasteners that fit into the one or more apertures; and inserting one of the fasteners into one of the apertures so as to push the tab away from the aperture. In one aspect, the implant can be implanted by a method of implantation that includes: obtaining an implant having one or more apertures and one or more tabs associated with each aperture; obtaining one or more fasteners that fit into the one or more apertures; inserting one of the fasteners into one of the apertures so as to push the tap away from the aperture; and inserting one of the fasteners into one of the apertures adjacent to the aperture already having the fastener so as to push the tab away from the aperture receiving the fastener and toward the first-inserted fastener. When being pushed back to the first-inserted fastener, the tab can be pressed against the first-inserted fastener, compressed against the first-inserted fastener, slid over the first-inserted fastener, or inserted into a slot on the first-inserted fastener. After the second-inserted fastener is installed, the tab can then automatically, such as by being flexibly resilient, press against, be compressed against, or cover the second-inserted fastener, or this can be performed by the medical practitioner after installation of the second-inserted fastener. As such, the tab can provide friction to two adjacent fasteners in two adjacent apertures. Alternatively, two adjacent apertures with a tab therebetween can receive fasteners at the same time. The tab can simultaneously be pushed toward the other fastener so as to provide friction coupling to both fasteners when installed. Optionally, the tab can be lifted out of the plane of the implant body during simultaneous installation. Also, a fastener can be partially inserted into an aperture, then a fastener can be inserted into an adjacent aperture separated by a tab, before the first fastener is fully inserted. The installation of the bone plate implant can include the use of fasteners being received into apertures with the tab then facilitating retention of the fastener in the aperture as described herein.

In one embodiment, the implant can be implanted by a method of implantation that includes: obtaining an implant having one or more apertures and one or more tabs associated with each aperture; obtaining one or more fasteners that fit into the one or more apertures; inserting one of the fasteners into one of the apertures so as to push the tab away from the aperture; and rotating a cam adjacent to the tab opposite of the aperture so as to cam the tab back toward or against the fastener. In one aspect, the implant can be implanted by a method of implantation that includes: obtaining an implant having one or more apertures and one or more tabs associated with each aperture; obtaining one or more fasteners that fit into the one or more apertures; obtaining one or more cams that fit into one or more apertures; inserting one of the fasteners into one of the apertures so as to push the tab away from the aperture; inserting one of the cams into one of the apertures adjacent to the aperture already having the fastener; and rotating the cam so as to push the tab away from the aperture receiving the cam and toward the first-inserted fastener. When being pushed back to the first-inserted fastener by rotating the cam, the tab can be pressed against the first-inserted fastener, compressed against the first-inserted fastener, slid over the first-inserted fastener, or inserted into a slot on the first-inserted fastener. After the cam is installed, the tab can then be pressed against, be compressed against, or cover the second-inserted fastener by rotation of the cam so as to move the tab. This can be performed by the medical practitioner after installation of the cam. Alternatively, two adjacent apertures with a tab therebetween can receive a fastener and cam at the same time, and then the cam can be rotated to press the tab against the fastener. Also, a fastener can be partially inserted into an aperture, then a cam can be inserted into an adjacent aperture separated by a tab, before the first fastener is fully inserted and then the cam can be rotated. The installation of the bone plate implant can include the use of fasteners being received into apertures with the tab then rotating the cam to press the tab against the fastener thereby facilitating retention of the fastener in the aperture as described herein.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references, patent applications, published patent applications, and patents recited herein are incorporated herein by specific reference in their entirety: U.S. patent application Ser. No. 11/145,372, filed Jun. 3, 2005 (PG-PUB 2006/0276901); U.S. patent application Ser. No. 11/241,143, filed Sep. 30, 2005 (PG-PUB 2006/0276899); U.S. patent application Ser. No. 11/299,395, filed Dec. 12, 2005 (PG-PUB 2007/0135827); U.S. patent application Ser. No. 11/351,665, filed Feb. 14, 2006 (PG-PUB 2006/0276902); U.S. patent application Ser. No. 11/404,938, filed Apr. 14, 2006 (PG-PUB 2007/0073399; U.S. Pat. No. 7,727,279); U.S. patent application Ser. No. 11/472,060, filed Jun. 21, 2006 (PG-PUB 2007/0156239; U.S. Pat. No. 7,879,099); U.S. patent application Ser. No. 11/638,652, filed Dec. 12, 2006 (PG-PUB 2007/0239160; U.S. Pat. No. 7,883,542); U.S. patent application Ser. No. 11/804,838, filed May 21, 2007 (PG-PUB 2008/0091269; U.S. Pat. No. 7,909,872); U.S. patent application Ser. No. 11/872,519, filed Jul. 12, 2007; U.S. patent application Ser. No. 12/583,660, filed Aug. 24, 2009; U.S. patent application Ser. No. 13/065,291, filed Mar. 18, 2011; and U.S. patent application Ser. No. 13/199,324, filed Aug. 26, 2011. Additionally, this application cross-references U.S. patent application Ser. Nos. 13/370,925 and 13/478,870, which applications and patents are incorporated herein by specific reference. This patent application also cross-references U.S. patent applications having attorney docket numbers Z1425.10002US02 and Z1425.10003US02 (Applicant hereby authorizes attorney to insert U.S. Serial Numbers when known), filed herewith, which are incorporated herein by specific reference in their entirety.

1. An implant comprising:
a body having one or more apertures; and
one or more tabs associated with the one or more apertures, each tab being movable with respect to the body and one of the apertures.

2. The implant of claim 1, comprising:
a first position with a first tab in a relaxed state adjacent to a first aperture; and
a second position with the first tab a strained state with a portion of the first tab bent away from or toward the first aperture.

3. The implant of claim 1, comprising:
a first position with a first tab in a relaxed state between at least a first aperture and a second aperture; and
a second position with the first tab a strained state with a portion of the first tab bent away from the first aperture and toward the second aperture.

4. The implant of claim 1, wherein the tab includes a base connected to the body, a stem connected to the base, and a head connected to the stem opposite of the base.

5. The implant of claim 4, wherein the head is wider than the stem.

6. The implant of claim 5, wherein the head has a first lobe defining a portion of a first aperture and as second lobe defining a portion of a second aperture.

7. The implant of claim 4, wherein the tab includes a groove through the head and at least a portion of the stem so as to form adjacent tab flaps.

8. The implant of claim 7, wherein the adjacent tab flaps include a first tab flap defining a portion of a first aperture and a second tab flap defining a portion of a second aperture.

9. The implant of claim 4, wherein the tab is integrated with the implant.

10. The implant of claim 4, wherein the tab is cut from the body of the implant.

11. The implant of claim 4, wherein first and second apertures include first and second slotted grooves adjacent to the stem.

12. The implant of claim 11, comprising slotted apertures at an end of the slotted groves opposite of the first and second apertures.

13. The implant of claim 4, comprising a wall defining first and second apertures and the tab therebetween.

14. The implant of claim 7, comprising a wall defining first and second apertures and the tab therebetween and the groove in the tab that forms the tab flaps.

15. The implant of claim 1, wherein the body is a unitary member with the tab formed therein, wherein at least one aperture defines a portion of the tab.

16. The implant of claim 1, wherein the body is a unitary member with the tab formed therein, and wherein at least two apertures define two opposite sides of a head of the tab.

17. The implant of claim 1, wherein the body is a unitary member with the tab formed therein, wherein at least two apertures define two opposite two sides of a head of the tab, and wherein an end groove extends between the two apertures and defines an end of the tab between the two sides.

18. The implant of claim 1, wherein the body is a unitary member with the tab formed therein, wherein at least two apertures define two opposite sides of a head of the tab, wherein an end groove extends between the two apertures and defines an end of the tab between the two sides, and wherein a slotted grove extends from each of the two apertures so as to form a stem of the tab.

19. The implant of claim 1, wherein the body is a unitary member with the tab formed therein, wherein at least two apertures define two opposite sides of a head of the tab, wherein an end groove extends between the two apertures and defines an end of the tab between the two sides, and wherein a slotted groove extends from each of the two apertures so as to form a stem of the tab, the tab has a base connected to a main body portion of the body, the stem is connected to the base, and the head is connected to the stem opposite of the base.

20. The implant of claim 1, wherein the body is a unitary member with the tab formed therein, wherein at least two apertures define two opposite sides of a head of the tab, wherein an end groove extends between the two apertures and defines an end of the tab between the two sides, and wherein a slotted groove extends from each of the two apertures so as to form a stem of the tab, the tab has a base connected to a main body portion of the body, the stem is connected to the base, and the head is connected to the stem opposite of the base, the apertures, end groove, and slotted grooves are defined by a continuous wall of the body.

21. A method of forming the implant of claim 1, the method comprising:
providing the body;
forming the one or more apertures in the body; and
forming the one or more tabs.

22. A method of forming the implant of claim 20, the method comprising:
providing the body; and
forming the apertures, end groove, and slotted grooves so as to form the tabs.

23. An implant system comprising:
the implant of claim 1; and
one or more fasteners or one or more cams dimensioned to fit in the one or more apertures.

24. The implant system of claim 23, wherein a first fastener includes a fastener head that presses against a first tab when inserted into an aperture that defines a surface of the first tab.

25. An implant system comprising:
the implant of claim 1;
one or more fasteners dimensioned to fit in a portion of the one or more apertures; and
one or more cams dimensioned to fit in a portion of the one or more apertures.

26. The implant system of claim 23, wherein a first fastener includes a first fastener head that moves a first tab when inserted into a first aperture that defines a surface of the first tab, and a first cam includes a second fastener head that presses against the first tab when inserted into a second aperture that defines a surface of the first tab and the first cam moves the first tab toward the first aperture when the first cam is rotated.

27. A method of mounting an implant to a bone, the method comprising:
providing the implant system of claim 23;
inserting a first fastener into a first aperture so as to move a head of the first tab; and
inserting the first fastener into the bone so as to fasten the implant to the bone.

28. The method of claim 27, comprising:
rotating a head of the first tab away from the first aperture toward a second aperture before or during inserting the first fastener into the first aperture; and
rotating the head of the first tab toward the first aperture before or during inserting a second fastener into the second aperture.

29. The method of claim 28, comprising:
pressing the head of the first tab against the first fastener so as to friction couple the first tab with the first fastener.

30. The method of claim 29, comprising:
pressing the head of the first tab against the first fastener and second fastener so as to friction couple the first tab with the first fastener and second fastener.

31. The method of claim 28, comprising:
covering at least a portion of the first fastener with the first tab.

32. The method of claim 31, comprising:
covering at least a portion of the first fastener and second fastener with the first tab.
